(19) United States
(12) Patent Application Publication
(10) Pub. No.: US 2009/0274621 A1
(43) Pub. Date: Nov. 5, 2009

(54) USE OF PYRENE TO CARRY NON-PEPTIDE AGENTS ACROSS THE BLOOD BRAIN BARRIER

(75) Inventors: Renee Wegrzyn, Washington, DC (US); Andrew Nyborg, Gaithersburg, MD (US); D. Roxanne Duan, Bethesda, MD (US); Alan Rudolph, Potomac, MD (US)

Correspondence Address: FOLEY AND LARDNER LLP;SUITE 500, 3000 K STREET NW, WASHINGTON, DC, 20007, US

(73) Assignee: Adlyfe, Inc

(21) Appl. No.: 12/363,221

(22) Filed: Jan. 30, 2009

Related U.S. Application Data
(60) Provisional application No. 61/038,595, filed on Mar. 21, 2008.

(51) Int. Cl. A61K 51/00; A61K 49/00; A61B 5/055; A61K 31/7088; A61K 31/015

(52) U.S. Cl. 424/1.65; 424/9.1; 424/9.3; 424/9.6; 514/44 R; 514/765

(57) ABSTRACT

Described are methods for delivering a non-peptide agent across the blood-brain barrier, comprising administering to a subject a conjugate comprising (i) a non-peptide agent and (ii) pyrene, and related detection and therapeutic method.

### RELATED APPLICATIONS

This application claims the benefit of priority to U.S. provisional application 61/038,595, filed Mar. 21, 2008, the entire contents of which are incorporated herein by reference in their entirety.

### FIELD OF THE INVENTION

The present invention relates generally to the field of delivering non-peptide agents across the blood-brain barrier (BBB). More specifically, the present invention relates to methods for delivering non-peptide agents, including detection and therapeutic agents, across the BBB using pyrene-agent conjugates.

### BACKGROUND OF THE INVENTION

The detection and treatment of neurological conditions is often difficult due to the impermeability of endogenous and exogenously administered components to the brain as a result of the blood-brain barrier (BBB). The BBB effectively isolates the brain from peripheral agents such as peptides, proteins, large macromolecules, non-peptidic molecules, ions, and water-soluble non-electrolytes. For example, it is generally accepted that charged or hydrophilic molecules do not cross the BBB, nor to molecules with a molecular weight greater than about 700 kDa. The BBB therefore impedes the delivery of detection and therapeutic agents that otherwise may be useful to detect or treat a wide variety of neurological conditions.

Prior attempts at effectively transporting agents to the brain have included conjugating agents to carrier moieties, using liposomal formulations, and using nanoparticles. Exemplary carrier moieties include naturally occurring polyamines (U.S. Pat. No. 5,670,477), carriers such as lysozyme, hemoglobin, cytochrome-c and substance-P (U.S. Pat. No. 5,604,198), and sugars (U.S. Pat. No. 5,260,308).

There remains a need, however, for methods, agents and kits for delivering non-peptide agents, including detection and therapeutic agents, across the BBB.

### SUMMARY OF THE INVENTION

In accordance with one embodiment, the invention provides a method for delivering a non-peptide agent across the blood brain barrier, comprising administering to a subject a conjugate comprising the non-peptide agent and pyrene. In some embodiments the non-peptide agent is a detection agent capable of identifying a protein or structure associated with a neurological disorder. In another embodiment, the non-peptide agent is a therapeutic agent useful in the treatment of a neurological disorder. In some embodiments, polynucleotides are used as detection and therapeutic agents, while in other embodiments, the therapeutic agents are small molecules. In some embodiments, the conjugate further comprises a detectable label. In some embodiments, the pyrene is a pyrene derivative, such as an alkylated pyrene, pyrene butyrate, PEGylated pyrene or pyrene-albumin, a pyrene derivative comprising a free carboxyl group and a pyrene derivative comprising a free amine group.

In accordance with another embodiment, the invention provides an in vivo method of detection comprising administering to a subject a conjugate comprising a non-peptide detection agent and pyrene, and detecting the non-peptide detection agent that is localized in a subject's brain. In one embodiment, the detection agent is capable of identifying a protein, such as amyloid protein, or a structure, such as an amyloid plaque, associated with a neurological condition by detecting the fluorescence of pyrene. In some embodiments, the conjugate further comprises a detectable label, such as a fluorophore, MRI contrast agent, ion emitter, or a radioactive label.

In other embodiments, the invention provides a method for treating neurological disorders. The method comprises administering to a subject a conjugate comprising a non-peptide therapeutic agent and pyrene. In one embodiment the non-peptide agent is an anti-amyloid agent.

### DETAILED DESCRIPTION

Before particular embodiments of the invention are described and disclosed, it is to be understood that the particular materials, methods and compositions described herein are presented only by way of examples, and are not limiting of the scope of the invention. The technical and scientific terms used herein have the meanings commonly understood by one of ordinary skill in the art to which the present invention pertains, unless otherwise defined. Publications and other materials setting forth known methodologies to which reference is made are incorporated herein by reference in their entireties as though set forth in full.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein “subject” denotes any animal in need of detection or therapeutic treatment, including humans and domesticated animals, such as cats, dogs, swine, cattle, sheep, goats, horses, rabbits, and the like. “Subject” also includes animals used in research settings, including mice and other small mammals. A typical subject may be at risk of a neurological condition, disease or disorder or suspected of suffering from such a condition, or may be desirous of determining risk or status with respect to a particular condition. As used herein, “therapeutic” treatment includes the administration of a therapeutic agent to treat an existing condition, to prevent a condition that the subject is at risk or developing, or for health maintenance.

As used herein, the phrase “therapeutically effective amount” means that drug dosage in a subject that provides the specific pharmacological response for which the drug is administered in a patient in need of such treatment. It is emphasized that a therapeutically effective amount will not always be effective in treating the conditions/diseases described herein, even though such dosage is deemed to be a therapeutically effective amount by those of skill in the art.

As used herein, the term “naturally occurring” or “native” with reference to an agent refer to agents (e.g., peptides, proteins and non-peptide small molecules, such as hormones) that are present in the body or recovered from a source that occurs in nature. A native agent may be modified either chemically or enzymatically, including post-translational modifications, including but not limited to, acetylation, glycosylation, phosphorylation, lipid conjugation, acylation and carbonylation.

As used herein, the term “synthetic” with reference to an agent (e.g., a small molecule) specifies that the agent is not naturally occurring, but may be obtained by other means such as chemical synthesis, biochemical methods, or recombinant methods.

As used herein, the terms “DNA” and “RNA” refer to heteropolymers of deoxyribonucleotides (bases adenine, guanine, thymine, cytosine) or ribonucleotides (bases adenine, guanine, uracil, cytosine), respectively, also referred to as or “polynucleotides.” Polynucleotides can be assembled chemically using a DNA (or RNA) synthesizer. DNA also may be obtained from synthetic cDNA fragments and short oligonucleotide linkers using a recombinant DNA expression system.

As used herein, “si-RNA” refers to synthetic or native double stranded RNA molecules. Exemplary si-RNA may be between 21-23 nucleotides in length.

As used herein, “RNA interference (RNAi)” refers to a mechanism for inhibiting gene expression at the stage of translation by hindering the transcription of specific genes. siRNA are useful in RNAi methods. For example, siRNA that comprise a nucleotide sequence complementary to that of the targeted RNA strand can be used to inhibit transcription. The RNAi pathway typically is initiated by an enzyme dicer, which cleaves long, double-stranded RNA (dsRNA) molecules into short fragments of 20-25 base pairs. One of the two strands of each fragment, often referred to as the guide strand, is incorporated into the RNA-induced silencing complex (RISC) and pairs with complementary sequences.

As used herein, “sh-RNA” refers to synthetic or native RNA sequences that make a tight-hairpin turn and are used to silence gene expression in RNA interference methods.

As used herein, “DNA decoy” refers to short segments of synthetic or native DNA which are used to disrupt intracellular gene expression.

As used herein “small molecule” means a molecule with a molecular weight of less than about 500-600 kDa.

As used herein, the terms “analog” and “derivative” of an agent mean analogs and derivatives of such agents that retain substantially similar functional activity or substantially the same biological function or activity as the reference agent, as described herein. “Substantially similar functional activity” and “substantially the same biological function or activity” each means that the degree of biological activity is within about 50% to 100% or more, within 80% to 100% or more, or within about 90% to 100% or more, of that biological activity demonstrated by the reference agent, when the biological activity of each agent is determined by the same procedure or assay. For example, an analog or derivative of an agent may exhibit the same biological activity as the referent agent qualitatively, although it may exhibit greater or lesser activity quantitatively. The suitability of a given analog or derivative of an agent can be verified by routine screening methods to confirm that the analog or derivative exhibits an activity of interest that is substantially similar to that of the referent agent. An analog or derivative may possess additional structural features and/or exhibit additional functional properties, such as PEGylated agents, which comprise a PEG moiety and may exhibit a longer circulating half-life in vivo.

As used herein, an “analog” of a drug may be a pro-drug that is converted in vivo to the active, drug form.

### A. Methods for Delivering Non-Peptide Agents Across the BBB

Applicant has discovered that pharmaceutically relevant non-peptide agents, e.g., detection and therapeutic agents, conjugated to a pyrene carrier show an enhanced ability to cross the blood-brain barrier (BBB) when administered to a subject.

In one embodiment, there is provided a method for delivering a non-peptide agent across the BBB that comprises administering to a subject a conjugate comprising (i) a non-peptide agent and (ii) pyrene. In some embodiments, the non-peptide agent is a detection or therapeutic agent. In specific embodiments, the non-peptide agent is a detection agent capable of identifying a target protein or structure associated with a neurological condition. In other embodiments, the non-peptide agent is a therapeutic agent useful in treating a neurological condition. As used herein, “capable of identifying” means that the non-peptide agent selectively and preferentially binds to the target protein or structure.

In some embodiments, the pyrene-conjugated active agent exhibits a permeability across the BBB that is substantially greater than that of the non-conjugated active agent, such as at least three, at least five, at least ten, at least fifteen, at least twenty times greater, or more, than that of the non-conjugated active agent. One measure of permeability across the BBB is the amount of conjugate that enters the brain relative to the amount that was injected and relative to the amount that enters other tissues (% IDI). In some embodiments, the pyrene-conjugate has an octanol/water partition coefficient between 1-10.

The conjugate may be formulated in any composition suitable for administration to a subject, such as a composition comprising the conjugate and a pharmaceutically acceptable carrier. The conjugate may be administered by any suitable means, including by intranasal, intravenous, intraperitoneal, intraarterial, intramuscular, subcutaneous, oral, buccal, or transdermal, administration, and may be formulated accordingly. For example, the pharmaceutically acceptable carrier may be a liquid, so that the composition is adapted for parenteral administration, or may be solid, i.e., a capsule shell plus vehicle, a tablet, a pill and the like, formulated for oral administration. Alternatively, the pharmaceutically acceptable carrier may be in the form of a nebulizable liquid or solid so that the composition is adapted for inhalation. Pharmaceutically acceptable carriers are known in the art, and may include, without limitation, dissolution or suspension agents such as water or a naturally occurring vegetable oil like sesame, peanut, or cottonseed oil or a synthetic fatty vehicle like ethyl oleate or the like. Buffers, preservatives, antioxidants, binders, excipients, disintegrating agents, lubricants, sweetening agents and flavoring agents may also be included in the composition.

In the methods described herein, one or more conjugates comprising the same or different detection agents, therapeutic agents, pyrene moities and/or labels may be used, with each conjugate provided in the same composition or in one or more different compositions that may be administered simultaneously or sequentially by the same route or by one or more different routes.

The rate of localization and clearance or degradation of a conjugate can be assessed experimentally, for example, by administering the conjugates to mice and sacrificing them for analysis at different times post-administration, such as at time periods including 2 minutes, 10 minutes, 30 minutes, 60 minutes, 1 hour, 2 hours, 3 hours, 4 hours, 5 hours, 6 hours, or longer, post-administration.

The non-toxicity of the conjugates can be verified experimentally, for example, using in vitro assays and in vivo rodent toxicity studies that are known in the art.

### B. Non-Peptide Agents

The nature of the non-peptide agent is not limited, other than not being a peptide (e.g., not comprising amino acid residues). As noted above, detection and therapeutic agents are useful in accordance with the methods described herein, including detection agents useful for detecting neurological conditions and therapeutic agents useful for treating neurological conditions. A number of such agents are known in the art. Agents include non-peptide macromolecules and non-peptide small molecules. Agents may be native or naturally occurring agents, or may be synthetic agents synthesized using chemical or biochemical methods or obtained through recombinant means. The following lists are exemplary only, and not limiting of the scope of the invention.

### Detection Agents:

Exemplary detection agents include those that are capable of identifying a protein or structure associated with a neurological condition. For example, a variety of neurological conditions are associated with a specific structural form of a protein (e.g., a “misfolded protein” or a self-aggregated protein), while the protein in a different structural form (e.g., a “normal protein”) is not harmful. In many cases, the normal protein is soluble, while the misfolded protein forms insoluble aggregates. Examples of such insoluble proteins include prions in transmissible spongiform encephalopathy (TSE); AP-peptide in amyloid plaques of Alzheimer's disease (AD), cerebral amyloid angiopathy (CAA), and cerebral vascular disease (CVD); α-synuclein deposits in Lewy bodies of Parkinson's disease, tau in neurofibrillary tangles in frontal temporal dementia and Pick's discasc; superoxide dismutase in amylotrophic lateral sclerosis; and huntingtin in Huntington's disease.

Exemplary detection agents selectively bind to proteins or structures that may be present in the brain and associated with neurological conditions, such as misfolded Aβ protein or Aβ plaques associated with Alzheimer's Disease, or other misfolded proteins or structures associated with other neurological disorders, as exemplified above. In other embodiments, the non-peptide agent specifically binds to a target protein or structure associated with other neurological conditions, such as stroke, cerebrovascular disease, epilepsy, and benign and cancerous brain tumors such as glioblastoma's, pituitary tumors, or meningiomas.

In some embodiments, the detection agent comprises a detectable label. Detectable labels include fluorescent agents (e.g., fluorophores, fluorescent semiconductor nanocrystals), phosphorescent agents, chemiluminescent agents, chromogenic agents, quenching agents, dyes, radionuclides, metal ions, metal sols, ligands (e.g., biotin, streptavidin haptens, and the like), enzyme substrates, enzyme cofactors (e.g., NADPH), enzyme inhibitors, scintillation agents, inhibitors, magnetic particles, oligonucleotides, polynucleotides, and other moieties known in the art.

Conjugates that do not target a specific protein or structure in the brain may find application in various in vitro and in vivo imaging studies, such as, in vivo measurements of interstitial volumes. Conjugates comprising pyrene and image contrast agents, for example, MRI, and PET scan agents, exhibit enhanced permeability across the BBB, thus improving the overall sensitivity of these diagnostic methods.

In another embodiment, the pyrene non-peptide conjugate further comprises a detectable label. In these embodiments, the specificity of the non-peptide agent for a target protein or structure achieves selective labeling of such proteins or structures. In one embodiment, the non-peptide agent of the labeled conjugate selectively targets an amyloid protein associated with Alzheimer's disease and achieves selective detection of this pathological condition.

As mentioned above, fluorescent labels are commonly used for imaging. Fluorophores and quenchers may include the following agent (or fluorophores and quenchers sold under the following tradenames): 1,5 IAEDANS; 1,8-ANS; umbelliferone (e.g., 4-Methylumbelliferone); acradimum esters, 5-carboxy-2,7-dichlorofluorescein; 5-Carboxyfluorescein (5-FAM); 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Carboxy-X-rhodamine (5-ROX); Cascade Blue™; Cascade Yellow; Catecholamine; CCF2 (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiIC18(5)); DIDS; Dihydrorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); a fluorescent protein (e.g., GFP (S65T); GFP red shifted (rsGFP); GFP wild type, non-UV excitation (wtGFP); GFP wild type, UV excitation (wtGFP); and GFPuv); Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; luminol, Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodamineIsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

Agents may include derivatives of fluorophores that have been modified to facilitate conjugation to another reactive molecule. As such, agents may include amine-reactive derivatives such as isothiocyanate derivatives and/or succinimidyl ester derivatives of the agent.

In embodiments for in vivo detection, agents useful for in vivo detection can be used. For example, agents useful for magnetic resonance imaging (MRI) and positron emission tomography (PET) as well as chemiluminescent agents can be used.

In one embodiment, the label is a PET or an MRI image contrast agent. Although MRI was initially hoped to provide a means of making definitive diagnoses noninvasively, the addition of contrast agents in many cases improves the sensitivity and/or specificity towards the tissue being imaged. MRI contrast agents can include positive or negative agents. Positive agents generally include paramagnetic molecule or short-T1 relaxation agents, although the combination of the two are also used. Exemplars of paramagnetic, positive GI contrast agents include ferric chloride, ferric ammonium citrate, and gadolinium-DTPA (with and without mannitol). Short T1 relaxation time contrast agents include mineral oil, oil emulsions, and sucrose polyester. Diamagnetic agents are used as negative contrast agent, for example, a mixture of kaolin and bentonite. Another diamagnetic contrast agent is suspension of a barium sulfate. Additionally, perfluoro chemical agents, such as Perfluoroctylbromide (PFOB) can also be used as a negative MRI contrast agent. Superparamagnetic agents can be used as oral negative MRI contrast agents. Compounds such as magnetite albumin microspheres, oral magnetic particles (Nycomed A/S, Oslo, Norway), and superparamagnetic iron oxide (AMI121, Advanced Magnetics, Cambridge, Mass.) are generally used. These compounds contain small iron oxide crystals approximately 250 to 350 angstroms in diameter and are mixtures of Fe2O3 and Fe3O4.

In another embodiment, the agent or label is a radioactive agent. For example, the agent may provide positron emission of a sufficient energy to be detected by machines currently employed for this purpose. One example of such an entity comprises oxygen-15 (an isotope of oxygen that decays by positron emission). Another example are compounds having fluorine-18 such as F-18 fluoro-L-dopa (FDOPA), F-18 fluorotyrosine (FTYR), fluorodeoxyglucose (FDG) as well as compounds containing C₁₁ atoms, (e.g., C-11 methionine (MET).

Agents may include oligonucleotides. For example, the agent may comprise an oligonucleotide tag which may be detected by known methods in the art (e.g., amplification assays such as PCR, TMA, b-DNA, NASBA, and the like).

Where the agent or label is a fluorophore, one or more characteristics of the fluorophore may be used to assess the state of the labeled conjugate. For example, the excitation wavelength of the fluorophore may differ based on whether the conjugate is bound or free. In some embodiments, the emission wavelength, intensity, or polarization of fluorescence also may vary based on the state of the conjugate.

### Therapeutic Agents:

Exemplary therapeutic agents include non-peptide macromolecules and small molecules. Exemplary non-peptide macromolecules include RNA, RNA decoys, si-RNA, sh-RNA, small RNA, ribosomal RNA, t-RNA, DNA, DNA decoys, introns, exons, full and partial gene sequences, promoters, and enhancers. Exemplary small molecules include non-peptide neurotransmitters, anti-psychotic agents, anti-epileptic agents, cholinergic agents, anticholinesterase agents, catecholamines, adrenergic receptor agonists and antagonists, small molecule inhibitors of Aβ, antimuscarinic compounds, opiates, antipsychotic compounds, and other neuroactive non-peptide drugs.

The following is a non-limiting list of neurologically active compounds, that fall within the scope of the aforementioned general categories.

a. Cholinergic Agonists

Suitable cholinergic agonists include, but are not limited to, choline chloride, acetylcholine chloride, methacholine chloride, carbachol chloride, bethanechol chloride, pilocarpine, muscarine, arecoline and the like.

b. Anticholinesterase Agents

Suitable anticholinesterase compounds include carbaril, physostigmine, neostigmine, edrophonium, pyridostigmine, demecarium, ambenonium, tetrahydroacridine and the like.

c. Catecholamines and Other Sympathomimetic Neurologically Active Compounds

Suitable catecholamines and sympathomimetic drugs include the subclasses of endogenous catecholamines, beta-adrenergic agonists, alpha-adrenergic agonists and other miscellaneous adrenergic agonists. Within the subclass of endogenous catecholamines, suitable examples include epinephrine, norepinephrine, dopamine and the like. Suitable examples within the subclass of β-adrenergic agonists include, but are not limited to, isoproterenol, dobutamine, metaproterenol, terbutaline, albuterol, isoetharine, pirbuterol, bitolterol, ritodrine and the like. The subclass of alpha-adrenergic agonists can be exemplified by methoxamine, phenylephrine, mephentermine, metaraminol, clonidine, guanfacine, guanabenz, methyldopa and the like. Other miscellaneous adrenergic agents include, but are not limited to, amphetamine, methamphetamine, methylphenidate, pemoline, ephedrine and ethylnorepinephrine and the like.

d. Adrenergic Receptor Antagonists

Adrenergic receptor antagonists include the subclasses of α-adrenergic receptor antagonists and β-adrenergic receptor antagonists. Suitable examples of neurologically active compounds that can be classified as alpha-adrenergic receptor antagonists include, but are not limited to, phenoxybenzamine and related haloalkylamines, phentolamine, tolazoline, prazosin and related drugs, ergot alkaloids and the like. Both selective and nonselective β-adrenergic receptor antagonists can be used, as can other β-adrenergic receptor antagonists.

e. Antimuscarinic Neurologically Active Compounds

Antimuscarinic drugs are exemplified by the group consisting of atropine, scopolamine, homatropine, belladonna, methscopolamine, methantheline, propantheline, ipratropium, cyclopentolate, tropicamide, pirenzepine and the like.

f. Compounds that Act at the Neuromuscular Junction and Autonomic Ganglia

Exemplary neurologically active compounds that can be classified as compounds that act at the neuromuscular junction and autonomic ganglia include, but are not limited to tubocurarine, alcuronium, β-erythroidine, pancuronium, gallamine, atracuriam, decamethonium, succinylcholine, nicotine, labeline, tetramethylammonium, 1,1-dimethyl-4-phenylpiperazinium, hexamethonium, pentolinium, trimethaphan and mecamylamine, and the like.

g. Non-Peptide Neurotransmitters

Non-peptide neurotransmitters include the subclasses of neutral amino acids, such as glycine and gamma-aminobutyric acid, and acidic amino acids such as glutamate, aspartate, and NMDA receptor antagonist-MK801 (Dizocilpine Maleate). Other suitable non-peptide neurotransmitters include acetylcholine and the subclass of monoamines, such as dopamine, norepinephrine, 5-hydroxytryptamine, histamine, and epinephrine.

h. Antiepileptic Neurologically Active Compounds

Exemplary antiepileptic drugs include, but are not limited to, hydantoins such as phenyloin, mephenyloin, and ethotoin; anticonvulsant barbiturates such as phenobarbital and mephobarbital; deoxybarbiturates such as primidone; iminostilbenes such as carbamazepine; succinimides such as ethosuximide, methsuximide, and phensuximide; valproic acid; oxazolidinediones such as trimethadione and paramethadione; benzodiazepines and other antiepileptic agents such as phenacemide, acetazolamide, and progabide.

i. Agents for Treating of Neurological Disorders Associated with Movement

Neurologically active compounds that are effective in the treatment of, for example, Parkinsonism and other movement disorders include, but are not limited to, dopamine, levodopa, carbidopa, amantadine, baclofen, diazepam, dantrolene, dopaminergic agonists such as apomorphine, ergolines such as bromocriptine, pergolide, and lisuride, and anticholinergic drags such as benztropine mesylate, trihexyphenidyl hydrochloride, procyclidine hydrochloride, biperiden hydrochloride, ethopropazine hydrochloride, and diphenhydramine hydrochloride.

j. Opiates

Exemplary opioid analgesics include, but are not limited to morphine and related opioids such as levorphanol and congeners; meperidine and congeners such as piperidine, phenylpiperidine, diphenoxylate, loperamide, and fentanyl; methadone and congeners such as methadone and propoxyphene; pentazocine; nalbuphine; butorphanol; buprenorphine; meptazinol; opioid antagonists such as naloxone hydrochloride; and centrally active antitussive agents such as dextromethorphan.

k. Antipsychotic Compounds

Neurologically active compounds that can be used to treat depression, anxiety or psychosis include, but are not limited to, phenothiazines, thioxanthenes, dibenzodiazepines, butyrophenones, diphenylbutylpiperidines, indolones, and rauwolfia alkaloids. Mood alteration drugs include, but are not limited to, tricyclic antidepressants (which include tertiary amines and secondary amines), atypical antidepressants, and monoamine oxidase inhibitors. Examples of suitable drugs that are used in the treatment of anxiety include, but are not limited to, benzodiazepines.

l. Neuroactive Non-Peptide Drugs

Neurologically active agents useful in the present conjugate include neuroactive nonprotein drugs, such as neurotransmitter receptors, muscarinic agonists, serotonic receptors, agents, and actions; thiazole-containing 5-hydroxytryptamine-3 receptor antagonists; acidic amino acids as probes of glutamate receptors and transporters; L-2-(carboxycyclopropyl)glycines; and N-Methyl-D-aspartic acid receptor antagonists and other pharmacological targets of, for example, Alzheimer's disease.

m. Anti-Amyloid Agents

“Anti-amyloid agents” or “anti-amyloidogenic agents,” are agents which, directly or indirectly, inhibit proteins from aggregating and/or forming amyloid plaques or deposits and/or promotes disaggregation or reduction of amyloid plaques or deposits, and are useful as agents in the methods described herein.

Anti-amyloid agents include chelating agents (e.g., chelating agents for transition metals such as copper and iron such as tridentate iron chelators), diketones (e.g., beta-diketones), 2-pyridoxal isonicontinyl hydrazone analogues, tachypyridine, clioquinol, ribonucleotide reductase inhibitor chelators, 2,3-dihydroxybenzoic acid, Picolinaldehyde, Nicotinaldehyde, 2-Aminopyridine, 3-Aminopyridine, topical 2-furildioxime, n-Butyric acid, Phenylbutyrate, Tributyrin, suberoylanilide hydroxamic acid, 6-cyclohexyl-1-hydroxy-4-methyl-2(1H)-pyridinone, rilopirox, piroctone, benzoic acid-related chelators, salicylic acid, nicotinamide, Clioquniol, heparin sulfate, trimethylamine N-oxide (TMNO), polyethylene glycol (PEG), copper cations (e.g., Cu⁺⁺), dimethylsulfoxide (DMSO), and Dexrazoxane.

Anti-amyloid agents also include agents generally referred to in the art as “amyloid busters” or “plaque busters.” These include drugs which are peptidomimetic and interact with amyloid fibrils to slowly dissolve them. “Peptidomimetic” means that a biomolecule mimics the activity of another biologically active peptide molecule. “Amyloid busters” or “plaque busters” also include agents which absorb co-factors necessary for the amyloid fibrils to remain stable.

Anti-amyloid agents also include dopamine, tannic acid, triazine, levodopa, pergolide, bromocriptine, selegiline, glucosamine or analogs thereof (e.g., 4-deoxy-D-glucosamine or 4-deoxy-acetylglucosamine), tetrapyrroles, nordihydroguaiaretic acid (NDGA), polyphenols (e.g., myricetin (Myr), morin (Mor), quercetin (Qur), kaempferol (Kmp), (+)-catechin (Cat), (−)-epicatechin (epi-Cat)), rifampicin (RIF), tetracycline (TC), small molecule sulfonic acids (e.g., polyvinylsulfonic acid and 1,3,-propanedisulfonic acid), small molecule sulphonates and sulfates (e.g., ethanesulfphonate, 1-propanesulphonate, 1,2-ethanedisulphonate, 1,3-propaendisulphonate, 1,4-butanedisulphonate, 1,5-propanedisulphonate, 1,6-hexanedisulphonate, poly(vinylsulphonate), 1,2-ethanediol disulphate, 1,3-propanediol disulphate, and 1,4-butanediol disulphate), cyclohexanehexyl (e.g., epi-cyclohexanehexyl, scyllo-cyclohexanehexyl, and myo-cyclohexanchexyl), β-sheet breaker peptide (iAβ5), nicotine, or salts, acids, or derivatives thereof.

Those skilled in the art will recognize that many of the non-peptide agents described above as exemplary detection agents also are useful as therapeutic agents, and that many of the non-peptide agents described above as exemplary therapeutic agents also are useful as detection agents. Thus, these descriptors are in no way limiting.

### C. Pyrene

The pyrene can be pyrene or any pyrene derivative or analog that, when conjugated to a non-peptide agent improves the permeability of the agent across the BBB.

Pyrene consists of four fused benzene rings:

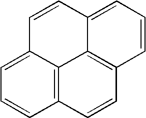

By “pyrene” derivative or analog is meant a molecule comprising the four fused benzene rings of pyrene, wherein one or more of the pyrene carbon atoms is substituted or conjugated to a further moiety. Exemplary pyrene derivatives include alkyl pyrenes, wherein one or more of the pyrene carbon atoms is substituted with a linear or branched, substituted or unsubstituted, alkyl, alkenyl, alkynyl or acyl group, such as a C₁-C₂₀, linear or branched, substituted or unsubstituted alkyl, alkenyl, alkynyl or acyl group, where the group may be substituted with, for example, a moiety including an O, N or S atom (e.g., carbonyl, amine, sulfhydryl) or with a halogen. In some embodiments the pyrene derivative includes one or more free carboxyl groups and/or one or more free amine groups, each of which may be directly attached to a pyrene carbon atom or attached to any position on a linear or branched, substituted or unsubstituted, alkyl, alkenyl, alkynyl or acyl group as described above, such as being attached at a carbon atom that is separated from a pyrene carbon by 1 or more, such as 1 to 3, 1 to 5, or more, atoms. In some embodiments, the pyrene is substituted with one or more acetic acid moieties and/or one or more ethylamine moieties. In some embodiments, the pyrene derivative is substituted with a single methyl, ethyl, propyl or butyl group. In some embodiments, the pyrene is substituted with a short chain fatty acid, such as pyrene butyrate. In another embodiment, the pyrene is conjugated to albumin, transferrin or an Fc fragment of an antibody. In some embodiments, the substituent is attached to pyrene through a carbon-carbon linkage, amino group, peptide bond, ether, thioether, disulfide, or an ester linkage.

Pyrene derivatives can be made by methods known in the art. For example, substituted pyrenes may be used to attach fatty acids to the tetracyclic scaffold. Suitable reagents, including functionalized alkyl derivatives of pyrene, and derivatizing reactions are known in the art. For example amino pyrene can be reacted with 1,4-butanedioic acid methyl ester to yield a butanoic acid derivative of pyrene. Alternatively, 1-thiocyanato pyrene can be reacted with 4-aminobuatnoic acid methyl ester to yield a thio-substituted butanoic acid derivative of pyrene. Yet other alternative reactions include reacting pyrene boronic acid and a substituted fatty acid to yield fatty acid derivatives of pyrene.

In other embodiments, the pyrene derivative is PEGylated pyrene, i.e, pyrene conjugated to polyethylene glycol (PEG). Such pyrene derivatives may exhibit a longer circulating half-life in vivo. In other embodiments, the pyrene derivative is pyrene conjugated to albumin (“albumin-pyrene”).

In some embodiments, the pyrene derivative exhibits reduced toxicity as compared to pyrene. In some embodiments, the pyrene derivative exhibits an increased circulating half-life in vivo as compared to pyrene, such as PEGylated pyrene discussed above. In some embodiments, the pyrene derivates exhibits even greater increased permeability across the BBB as compared to pyrene, such as albumin conjugated pyrene. In some embodiments, the pyrene derivative has an octanol/water partition coefficient between 1-10.

### D. Conjugates

The non-peptide active agent can be conjugated to pyrene by any means known in the art including chemical (covalent) conjugation. In some embodiments, the non-peptide agent is directly conjugated to pyrene. In other embodiments, the non-peptide agent is conjugated to pyrene through a linker. Compounds used as linkers are well known in the art, and include optionally substituted C₁-C₂₀ alkyl groups, alkanoic acids, alkenoic acids, alkynoic acids, alkoxide groups, aminoalkanoic acids, alkyl amines, alkoxy groups, bifunctional imido esters, glutaraldehyde, ethylene oxide polymers (PEG), optionally substituted aryl groups, alkynyl pyridyl, alkynyl bipyridyl, phthalic acid, malic acid and maleic acid, N-hydroxysuccinimide esters, hetero-bifunctional reagents and group specific-reactive agents such as the maleimido moiety, dithio moiety (SH) and carbodiimide moiety.

Conjugates may be formed by chemical synthesis or bioengineering methods, such as methods including expressing pyrene in living organisms together with the agent. Such bioengineering methods include direct engineering of synthetic biological processes or evolution and screening for pyrene-agent conjugate combinations.

In some embodiments, the non-peptide agent is conjugated to a single pyrene moiety. In other embodiments, the non-peptide agent is conjugated to two or more pyrene moieties. When the non-peptide agent is conjugated to two or more pyrene moieties, each pyrene moiety may be conjugated to the agent (directly or through a linker), and the pyrene moieties may be conjugated to the agent in close proximity to each other, or at spaced apart or distant positions on the non-peptide agent. In other embodiments, one or more pyrene moieties is conjugated (directly or through a linker) to one or more pyrene moieties, at least one of which is conjugated, directly or through a linker, to the agent. In any of these embodiments, the two or more pyrene moieties may be the same or different, such as being the same or different pyrene derivatives.

Regardless of the position(s) of the pyrene moiety(ies), the conjugate may exhibit enhanced permeability of the agent across the BBB.

In some embodiments, the conjugates are labeled with pyrene such that they are capable of forming pyrene excimers. That is, the non-peptide agents are conjugated to pyrene moieties in such as way as to permit excimer formation between pyrene moieties conjugated to the same or different molecules of non-peptide agent, as may be desired. In accordance with these embodiments, two or more pyrene moieties may be conjugated to the same non-peptide agent molecule so as to permit excimer formation by interaction between pyrene moieties on the same non-peptide agent molecule, such as may be associated, for example, with a specific conformation of the non-peptide agent. Alternatively, the excimer formation may be due to interaction between pyrene moieties on different non-peptide agent molecules, such as may be associated, for example, with localization, binding and/or interaction between the non-peptide agent molecules.

In some embodiments different pyrene derivatives are used, at least one of which includes one or more free carboxyl groups (such as an acetic acid moiety) and at least one of which includes one or more free amine groups (such as an ethylamine moiety), as discussed above. In accordance with this embodiment, interactions between the free carboxyl group(s) on one pyrene derivative and the free amine group(s) on another pyrene derivative may stabilize interactions between the pyrene derivatives, such as via the formation of a salt bridge, and may stabilize the excimer forming adducts and/or maximize excimer fluorescene. In accordance with these embodiments, two or more different pyrene derivatives may be conjugated to the same non-peptide agent molecule, such as to stabilize excimer formation by interaction between different pyrene derivatives on the same non-peptide agent molecule. Alternatively, one pyrene derivative may be conjugated to one non-peptide agent molecule and a different pyrene derivative may be conjugated to a different non-peptide agent molecule, such as to stabilize excimer formation by interaction between the different non-peptide agent molecules.

In some embodiments, the conjugate is labeled with a detectable label. In embodiments where the non-peptide agent is a detectable label, the detectable label may offer improved detection or detection under additional conditions. In embodiments where the non-peptide agent is a therapeutic agent, the detectable label may offer detection in addition to the therapy offered by the therapeutic agent.

As used herein, a “detectable label” includes any moiety that can be detected. Suitable detectable labels include those exemplified above with respect to non-peptide detection agents, as well as fluorescent proteins, enzymes, and other peptide-based detection moieties known in the art. Fluorescent proteins may include green fluorescent proteins (e.g., GFP, eGFP, AcGFP, TurboGFP, Emerald, Azami Green, and ZsGreen), blue fluorescent proteins (e.g., EBFP, Sapphire, and T-Sapphire), cyan fluorescent proteins (e.g., ECFP, mCFP, Cerulean, CyPet, AmCyan1, and Midoriishi Cyan), yellow fluorescent proteins (e.g., EYFP, Topaz, Venus, mCitrine, YPet, PhiYFP, ZsYellow1, and mBanana), and orange and red fluorescent proteins (e.g., Kusabira Orange, mOrange, dTomato, dTomato-Tandem, DsRed, DsRed2, DsRed-Express (T1), DsREd-Monomer, mTangerine, mStrawberry, AsRed2, mRFP1, JRed, mCherry, HcRed1, mRaspberry, HcRed-Tandem, mPlum and AQ143).

Where the agent or label is a fluorophore, one or more characteristics of the fluorophore may be used to assess the state of the labeled conjugate. For example, the excitation wavelength of the fluorophore may differ based on whether the conjugate is bound or free. In some embodiments, the emission wavelength, intensity, or polarization of fluorescence also may vary based on the state of the conjugate.

As used herein, a “fluorophore” is a chemical group that may be excited by light to emit fluorescence or phosphorescence. A “quencher” is an agent that is capable of quenching a fluorescent signal from a fluorescent donor. A first fluorophore may emit a fluorescent signal that excites a second fluorophore. A first fluorophore may emit a signal that is quenched by a second fluorophore. The conjugates disclosed herein may also undergo fluorescence resonance energy transfer (FRET).

E. In Vivo Detection with Non-Peptide Conjugates

Also provided are in vivo detection methods for detecting conjugate that has crossed the BBB and localized in the brain. As used herein, “localized in the brain” means has crossed the blood brain barrier, and includes localization in fluid surrounding the brain. In one embodiment, the method comprises (a) administering to a subject a conjugate comprising (i) a non-peptide detection agent and (ii) pyrene and (b) detecting detection agent that has localized in the brain of the subject. In some embodiments, the non-peptide detection agent specifically binds to a protein or structure localized in the brain. In some embodiments, the non-peptide detection agent specifically binds to a protein or structure localized in the brain and associated with a neurological condition, such as misfolded Aβ protein or Aβ plaques associated with Alzheimer's Disease, or other misfolded proteins or structures associated with other neurological conditions, as discussed above

In another embodiment, the method comprises (a) administering to a subject a conjugate comprising (i) a non-peptide agent and (ii) pyrene, wherein the conjugate is labeled with a detectable label, and (b) detecting conjugate that has localized in the brain of the subject.

As discussed above, the detection agent or label may be a fluorophore, an MRI contrast agent, ion emitter (PET), radioactive (scintillation counter), and the like. The conjugate can be detected by means suitable for detecting the detection agent or label, such as Fourier transform infra-red, ultra-violet, MRI, PET, scintillation counter, or fluorescence, light scattering, fluorescence resonance energy transfer (FRET), fluorescence quenching, and various chromatographic methods routinely used by one of ordinary skill in the art.

In some embodiments, the detecting step includes detecting pyrene excimer formation. An excimer is an adduct that is not necessarily covalent and that is formed between a molecular entity that has been excited by a photon and an identical unexcited molecular entity. The adduct is transient in nature and exists until it fluoresces by emission of a photon. An excimer represents the interaction of two fluorophores that, upon excitation with light of a specific wavelength, emits light at a different wavelength, which is also different in magnitude from that emitted by either fluorophor acting alone. It is possible to recognize an excimer (or the formation of an excimer) by the production of a new fluorescent band at a wavelength that is longer than that of the usual emission spectrum. An excimer may be distinguished from fluorescence resonance energy transfer since the excitation spectrum is identical to that of the monomer. The formation of the excimer is dependent on the geometric alignment of the fluorophores and is heavily influenced by the distance between them.

The formation of excimers may be detected by a change in optical properties. Such changes may be measured by known fluorimetric techniques, including UV, IR, CD, NMR, or fluorescence, among numerous others, depending upon the fluorophore attached to the probe. The magnitude of these changes in optical properties is directly related to the amount of conjugate that has adopted the structural state associated with the change, and is directly related to the amount of target protein or structure present.

The conjugates described herein also are useful in other in vivo detection methods. For example, the conjugates can be used to detect a target protein or structure (such as a specific conformation or state of self-aggregation) in any other in vivo site, such as any organ including the heart, lungs, liver, kidney, or any tissue. Specific areas of interest also may include vascular tissue or lymph tissue. The conjugates described herein also are useful in detecting and imaging a target protein or structure in intravial microscopy methods.

In some embodiments, conjugates comprising different fluorescent labels (such as, for example, GFP) can be used with the pyrene conjugates in FRET methodologies. Fluorescence resonance energy transfer (FRET) involves the radiationless transfer of energy from a “donor” fluorophore to an appropriately positioned “acceptor” fluorophore. The distance over which FRET can occur is limited to between 1-10 nm, and hence this technique is used to demonstrate whether two types of molecules, labeled with a donor-fluorophore and a receptor fluorophore, occur within 10 nm of each other. Measuring FRET by confocal imaging enables the intracellular locations of the molecular interaction to be determined.

FRET can occur when the emission spectrum of a donor fluorophore significantly overlaps (>30%) the absorption spectrum of an acceptor. The combination of CFP and YFP labelled fusion proteins has been widely used for FRET measurements in living cells. Other donor and acceptor fluorophore pairs which have been used for FRET include CFP and dsRED, BFP and GFP, GFP or YFP and dsRED, Cy3 and Cy5, Alexa488 and Alexa555, Alexa488 and Cy3, FITC and Rhodamine (TRITC), YFP and TRITC or Cy3.

Administration of such conjugates to a subject permits the detection of localized conjugate by the detection of the FRET signal.

F. Therapy with Non-Peptide Conjugates

Also provided are methods of treating neurological disorders that comprise delivering a therapeutic agent across the BBB. In one embodiment, the method comprises (a) administering to a subject a conjugate comprising (i) a non-peptide therapeutic agent and (ii) pyrene. In another embodiment, the conjugate is labeled with a detectable label, and the method further comprises detecting conjugate that has localized in the brain of the subject. In some embodiments, the non-peptide therapeutic agent is an anti-amyloid agent. In some embodiments, the method comprises administering a therapeutically effective amount of conjugate.

### EXAMPLES

The following examples provide further illustration of the invention without being limiting.

The following illustrates the ability of non-peptide-pyrene conjugates to cross the BBB. Similar methodology can be used to confirm the suitability of a given conjugate for use in accordance with the methods described herein, and/or to confirm that the conjugate exhibits enhanced permeability across the BBB as compared to the non-conjugated agent.

A non-peptide agent labeled with pyrene is administered to mice intranasally, at 10 μl liquid per administration (at concentrations of from 0.1 to 2.0 mg/ml) with an administration interval of a planned half of an hour, adjusted according to the condition of the animal after treatment. At the end of the treatment, mice are sacrificed and CSF and brains are extracted. (All mice are sedated by standard inhalation anaesthesia, Isofluran, Baxter). Cerebrospinal fluid is obtained by blunt dissection and exposure of the foramen magnum. Upon exposure, a Pasteur pipette is inserted to the approximate depth of 0.3-1 mm into the foramen magnum. CSF is collected by suctioning and capillary action until flow fully ceases. CSF is immediately frozen and kept at −80° C. until use.

After CSF sampling, the stomach, stomach content and the brains are rapidly removed. Brains are hemisected, and the right hemisphere of all mice are immersion fixed in freshly produced 4% Paraformaldehyde/PBS (pH 7.4) for one hour at room temperature, and transferred to a 15% sucrose/PBS solution for 24 hours to ensure cryoprotection. Thereafter, brains are frozen in liquid isopentane on the next day and stored at −80° C. until used for histological investigations. The other brain half is immediately shock frozen in liquid isopentane for future use.

Images are recorded from transgenic mice treated with the highest dose of conjugate and from control mice and from a transgenic vehicle control (e.g., the diluent used for the conjugate) to confirm that the conjugate crosses the blood-brain barrier (BBB), which it does.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with the true scope and spirit of the invention being indicated by the following claims.

What is claimed is:

1. A method for delivering a non-peptide agent across the blood-brain barrier, comprising administering to a subject a conjugate comprising:
(i) a non-peptide agent; and
(ii) pyrene.

2. The method of claim 1, wherein the non-peptide agent is a detection agent or a therapeutic agent.

3. The method of claim 1, wherein the non-peptide agent is a detection agent capable of identifying a protein or structure associated with a neurological condition.

4. The method of claim 1, wherein the non-peptide agent is a therapeutic agent useful in treating a neurological condition.

5. The method of claim 1, wherein the non-peptide agent is a polynucleotide detection or therapeutic agent.

6. The method of claim 1, wherein the non-peptide agent is a small molecule therapeutic agent.

7. The method of claim 1, wherein the conjugate further comprises a detectable label.

8. The method of claim 1, wherein the pyrene is a pyrene derivative.

9. The method of claim 1, wherein the pyrene derivative is selected from alkyl pyrene, amino pyrene, pyrene carboxylate, pyrene butyrate, albumin-pyrene, PEGylated pyrene, a pyrene derivative comprising a free carboxyl group and a pyrene derivative comprising a free amine group.

10. The method of claim 1, wherein conjugate exhibits enhanced permeability across the blood brain barrier as compared to the non-peptide agent.

11. An in vivo detection method comprising
(a) administering to a subject a conjugate comprising (i) a non-peptide detection agent and (ii) pyrene and
(b) detecting non-peptide detection agent that has localized in the brain of the subject.

12. The method of claim 11, wherein step (b) comprises detecting pyrene fluorescence.

13. The method of claim 11, wherein the detection agent is capable of identifying a protein or structure associated with a neurological condition.

14. The method of claim 11, wherein the detection agent selectively binds to an amyloid protein or structure associated with a neurological condition.

15. The method of claim 11, wherein the conjugate further comprises a detectable label.

16. The method of claim 15, wherein the label is selected from the group consisting of fluorophores, MRI contrast agents, ion emitters, and radioactive labels.

17. A method of treating a neurological disorder, comprising administering to a subject a conjugate comprising (i) a non-peptide therapeutic agent and (ii) pyrene.

18. The method of claim 17, wherein the non-peptide agent is a therapeutic agent useful in treating a neurological condition.

19. The method of claim 17, wherein the non-peptide agent is an anti-amyloid agent.

20. The method of claim 17, wherein the method comprises administering a therapeutically effective amount of the conjugate.
